(19) United States
(12) Patent Application Publication
(10) Pub. No.: US 2009/0093882 A1
(43) Pub. Date: Apr. 9, 2009

(54) SLIDING INTERBODY DEVICE

(76) Inventors: YoungHoon Oh, Montville, NJ (US); Mahmoud F. Abdelgany, Rockaway, NJ (US)

Correspondence Address: Rahman LLC, 10025 Governor Warfield Parkway, Suite 110, Columbia, MD, 21044, US

(21) Appl. No.: 11/869,007

(22) Filed: Oct. 9, 2007

(51) Int. Cl. A61F 2/44; A61B 17/70

(52) U.S. Cl. 623/17.11; 606/252

(57) ABSTRACT

A method of performing a surgical procedure and a sliding intervertebral implant includes an inner member that connects to an intervertebral space between two adjacent vertebrae, wherein the inner member includes a pair of arms spaced apart by a gap; an inclined plane configured in the gap; an arcuately-shaped back portion; and a fitting mechanism coupled below the pair of arms and the back portion, wherein the fitting mechanism includes a knob and neck. An outer member slidably attaches to the inner member, and includes a pair of curved arms spaced apart by a channel, wherein the pair of curved arms comprise an arcuate shape to match the arcuate shape of the back portion of the inner member, wherein the knob is configured to slide in the channel within a limitation of a pre-set curvature that is based on a pattern of a shape of the intervertebral space.

### BACKGROUND

1. Technical Field

The embodiments herein generally relate to medical devices, and, more particularly, to a sliding intervertebral implant used during orthopedic surgeries.

2. Description of the Related Art

Spinal fusion procedures may comprise the entire removal of the degenerated intervertebral disc between two adjacent vertebrae and the insertion of an implant within the intervertebral space. The implant may be positioned to maintain the spine alignment and the height and angle of the intervertebral space by pushing the vertebrae apart from each other, which helps in providing stability and proper maneuvering of the spine. Lastly, fusion material may be placed within the intervertebral space, which along with the body's natural cells, promotes bone formation. The fusion occurs between the endplates of the vertebrae.

A variety of implants of different configurations for intervertebral space have been developed to accomplish the spinal fusion surgeries. Some examples include spinal fusion cages, threaded bone dowels, stepped bone dowels, etc. The spinal fusion cages are mostly used as they are easy to handle. However, these cages offer some limitations. The spinal fusion cages generally do not maintain the spine alignment and the angle and height of the intervertebral space, thus the natural curvature of the spine may be changed. Also, it is typically very difficult to insert a spinal fusion cage into the vertebrae as they contain intricately combined parts. The wedge implants also suffer from certain drawbacks such as limited ability to prevent rotational forces between the vertebrae.

Most of these traditional intervertebral space implants are designed for either one-piece or multiple-pieces. For one-piece design, the implants are generally not accommodated to extend their surface contact in situ. Thus, to increase stability between two adjacent vertebrae, they generally have to be increased in size or inserted as a pair. For multiple assembly design, on the other hand, those parts are separated with rotational joints or expanded in heights (towards adjacent vertebral bodies). Generally, these tend to lack translation for all directions or have a limitation of rotation to increase the moment arm. Also, these devices are typically unable to sustain forces from the adjacent vertebrae and provide sufficient stability to the spine. Accordingly, there remains a need for a new intervertebral implant to restore motion in a patient's back in a controlled manner while permitting natural motion with stability.

### SUMMARY

In view of the foregoing, an embodiment herein provides a sliding intervertebral implant comprising an inner member adapted to connect to an intervertebral space between two adjacent vertebrae, wherein the inner member comprises a pair of arms spaced apart by a gap; an inclined plane configured in the gap; a back portion comprising an arcuate shape; and a fitting mechanism coupled below the pair of arms and the arcuate back portion, wherein the fitting mechanism comprises a knob and a neck. The implant further comprises an outer member slidably attached to the inner member, wherein the outer member comprises a pair of curved arms spaced apart by a channel, wherein the pair of curved arms comprise an arcuate shape to match the arcuate shape of the back portion of the inner member, wherein the knob is configured to slide in the channel within a limitation of a pre-set curvature, the pre-set curvature based on a pattern of a shape of the intervertebral space.

Preferably, the channel comprises an open end and a closed end, wherein the closed end fixes an end point of the inner member to slide with respect to the outer member. Additionally, the knob preferably comprises a width greater than a width of the neck. Also, the channel may comprise areas of multiple widths, wherein the multiple widths comprise a first width and a second width, and wherein the second width is greater than the first width. Furthermore, the knob may be adapted to slide with respect to the second width area of the channel. Moreover, the inner member and the outer member may be adapted to slide with respect to one another from a non-extended position to an extended position. Preferably, a top surface of the pair of arms of the inner member and a top surface of the outer member are planar with respect to one another in the non-extended position.

Another embodiment provides a sliding interbody device comprising a wedge-shaped first member adapted to connect to an intervertebral space between two adjacent vertebrae in a human body, wherein the first member comprises a pair of opposed arms and a knob, and an arcuate shaped second member configured to slidably attach to the first member, wherein the second member comprises a pair of opposed curved arms having a channel disposed therebetween, wherein each opposed curved arm comprises an arcuate top surface configured to match a contour of the first member, wherein the knob is configured to slide in the channel within a limitation of a pre-set curvature, the pre-set curvature based on a pattern of a shape of the intervertebral space, and wherein the channel comprises an open end and a closed end, wherein the closed end fixes an end point of the first member to slide with respect to the second member.

Furthermore, the pair of opposed arms of the first member may comprise a first arm perpendicular to a second arm, and wherein the first member may further comprise a gap separating the first arm from the second arm; an inclined plane configured in the gap and extending the length of the gap; a back portion comprising an arcuate shape; a bottom portion comprising an arcuate shape; and a neck outwardly extending from the bottom portion, wherein the knob outwardly extends from the neck, and wherein the knob is diametrically larger than the neck. Moreover, the channel may comprise areas of multiple widths, wherein the multiple widths comprise a first width and a second width, and wherein the second width is greater than the first width. Preferably, the pair of opposed curved arms of the second member comprises a first curved arm perpendicular to a second curved arm, wherein the channel comprises an arcuate shape and separates the first curved arm from the second curved arm, and wherein the channel comprises a pair of diametrically opposed grooves extending the length of the channel and located at a bottom portion of the channel.

Additionally, the knob may be adapted to align with the grooves of the channel. Furthermore, the first member and the second member may be adapted to slide in an arcuate path with respect to one another from a non-extended position to an extended position. Also, a top surface of the pair of opposed arms of the first member and a top surface of the second member are preferably planar with respect to one another in the non-extended position.

Another embodiment provides a method of performing a surgical procedure, wherein the method comprises engaging an intervertebral sliding implant in a non-extended position to a vertebral body, wherein the intervertebral sliding implant comprises a first member and a second member slidably attached to one another; adjusting the first member according to an intervertebral space between two adjacent vertebrae; and sliding the first member with respect to the second member within a limitation of a pre-set curvature to an extended position, wherein the pre-set curvature is based on a pattern of a second shape of the intervertebral space.

Preferably, the first member of the intervertebral sliding implant comprises an inclined plane adapted to fit a second shape of the intervertebral space; a curved back coupled to the inclined plane; a knob coupled to the curved back; and a neck coupled to the knob and the curved back of the first member. Furthermore, the second member of the intervertebral sliding implant preferably comprises a first curved arm; a second curved arm, wherein the first curved arm and the second curved arm are configured to accommodate the curved back of the first member; and a channel separating the first curved arm and the second curved arm, wherein the knob and the neck slide in the channel.

Additionally, the first member and the second member may be adapted to slide in an arcuate path with respect to one another from a non-extended position to an extended position. Moreover, the channel of the second member may comprise an extension adapted to accommodate the knob of the first member. Also, the channel may comprise an open end and a closed end, wherein the closed end fixes an end point of the first member to slide with respect to the second member.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a schematic diagram illustrating a side view of a sliding intervertebral implant having an inner member and an outer member in an extended position according to an embodiment herein;

FIGS. 2A through 2D are schematic diagrams illustrating a perspective view, a sectional view, a bottom view, and a back view, respectively, of the outer member of FIG. 1 according to an embodiment herein;

FIGS. 3A through 3D are schematic diagrams illustrating a perspective view, a sectional view, a front view, and a back view, respectively, of the inner member of FIG. 1 according to an embodiment herein;

FIGS. 4A through 4D are schematic diagrams illustrating a perspective view, a sectional view, a top view, and a back view, respectively, of the sliding intervertebral implant of FIG. 1 having the inner member and the outer member in a non-extended position according to embodiment herein;

FIG. 5 is a schematic diagram illustrating a perspective view of the sliding intervertebral implant of FIG. 1 having the inner member and the outer member in an extended position according to an embodiment herein; and

FIG. 6 is a process flow diagram illustrating a method of performing a surgical procedure according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

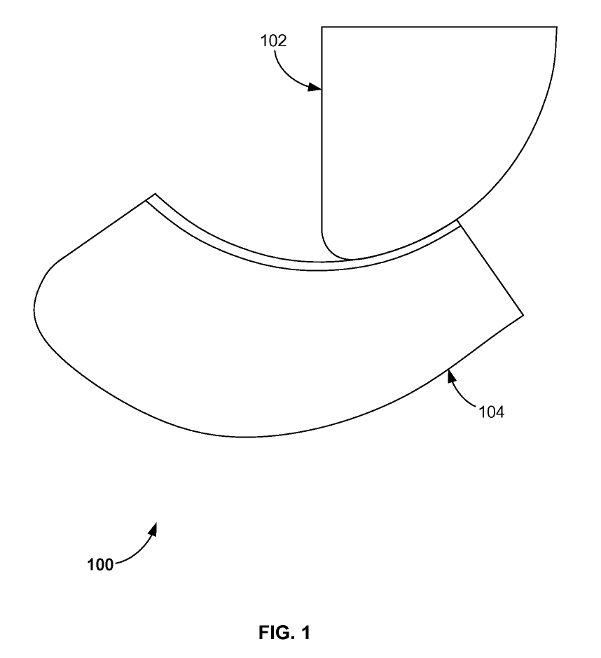

As previously mentioned, there remains a need for a new intervertebral implant to restore motion in a patient's back in a controlled manner while permitting natural motion with stability. The embodiments herein achieve this by providing an intervertebral implant that has two pieces that slide with respect to one another and moves from an extended position to a non-extended position. The first piece of the implant includes a slot configured to accommodate a correspondingly sized knob of the second piece to facilitate the sliding action. Referring now to the drawings, and more particularly to FIGS. 1 through 6, where similar reference characters denote corresponding features consistently throughout the figures, there are shown preferred embodiments.

FIG. 1 illustrates a side view of a sliding intervertebral implant 100 having an inner member 102 and an outer member 104 in an extended position according to an embodiment herein. The inner member 102 is positioned on the outer member 104. Both the inner member 102 and the outer member 104 preferably have a curved shape such that the inner member 102 is wedge-shaped and the outer member 104 is arcuate-shaped. These contours of the inner member 102 and outer member 104 are advantageous to adapt to the natural curvature of the spinal region of the human anatomy. The inner member 102 is in a sliding position. The curved shape of the outer member 104 helps the inner member 102 to easily slide through it.

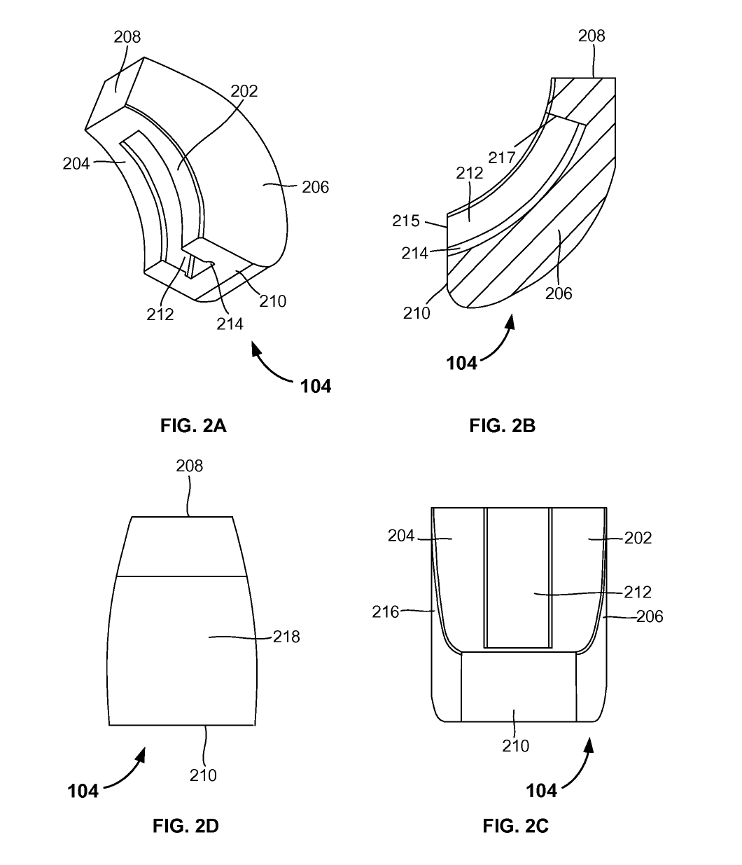

FIGS. 2A through 2D illustrate a perspective view, a sectional view, a bottom view, and a back view, respectively, of the outer member 104 of FIG. 1 according to an embodiment herein. The outer member 104 includes a first curved arm 202, a second curved arm 204, a first curved side 206 having a top portion 208, a bottom wall 210, a channel 212 with an extension 214, a second curved side 216, and a back side 218. FIG. 2A is the perspective view of the outer member 104 which shows the first curved arm 202 and the second curved arm 204 attached to the top portion 208. FIG. 2B is the sectional view of the outer member 104.

The channel 212 separates the first curved arm 202 and the second curved arm 204 from each other. The arms 202, 204 start from just below the top portion 208 and extends to the bottom wall 210. The channel 212 is spaced apart from the top portion 208 and extends to the bottom wall 210. A first end 215 of the channel 212 near the bottom wall 210 is open while the second end 217 near the top portion 208 is closed (best shown in FIG. 2B). The channel 212 has a first width defined by the gap between the first curved arm 202 and the second curved arm 204, and a second width defined by the extension 214 of the channel 212. The extension 214 may be larger than the width of the channel 212. The channel 212 may have the extension 214 throughout its bottom end. FIG. 2C is the bottom view of the outer member 104 which illustrates the first curved arm 202 with the first curved side 206 and the second curved arm 204 with the second curved side 216. FIG. 2D is the back view of the outer member 104 which shows the top portion 208, the bottom wall 210, and the back side 218. The curved shape of the first arm 202 and the second arm 204 support the curved shape of the inner member 102.

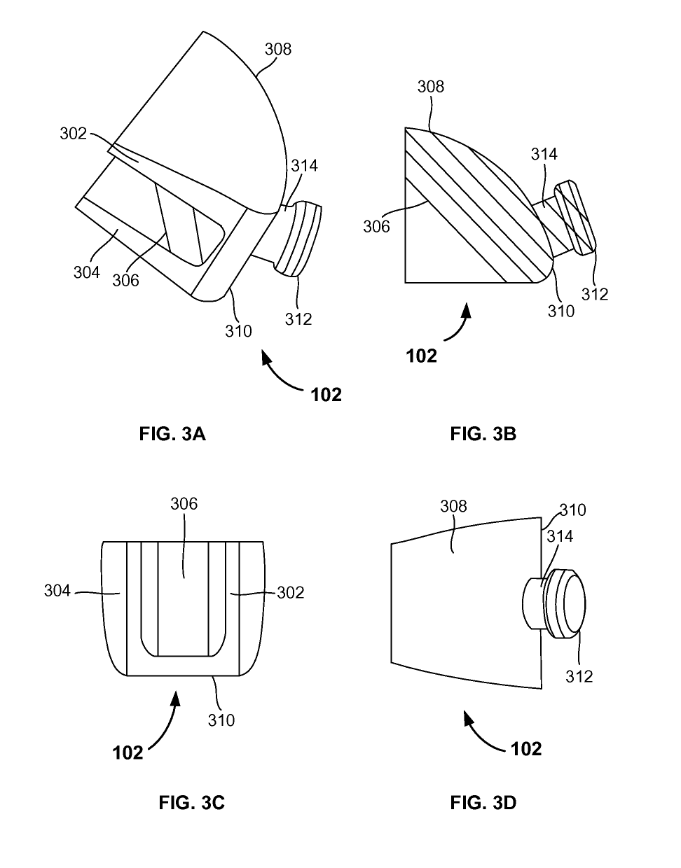

FIGS. 3A through 3D illustrate a perspective view, a sectional view, a front view, and a back view, respectively, of the inner member 102 of FIG. 1 according to an embodiment herein. The inner member 102 includes a first arm 302, a second arm 304, an inclined plane 306, a curved back 308, and an inner bottom portion 310 having a knob 312 with a neck 314. FIG. 3A is the perspective view of the inner member 102. The first arm 302 and the second arm 304 may be curved at their back side 308. The inclined plane 306 defines a channel/gap that separates the first arm 302 and the second arm 304 from each other.

FIG. 3B is the sectional view which shows the inclined plane 306, the curved back 308, and the inner bottom portion 310 having the knob 312 with the neck 314. FIG. 3C is the front view of the inner member 102, which illustrates the first arm 302, the second arm 304, the inclined plane 306, and the inner bottom portion 310. FIG. 3D is the back view which shows the curved back 308 and the inner bottom portion 310 having the knob 312 and the neck 314. The inclined plane 306 may be filled with fusion material (i.e., bone, bone morphogenetic protein (BMP), etc.) after insertion into the body.

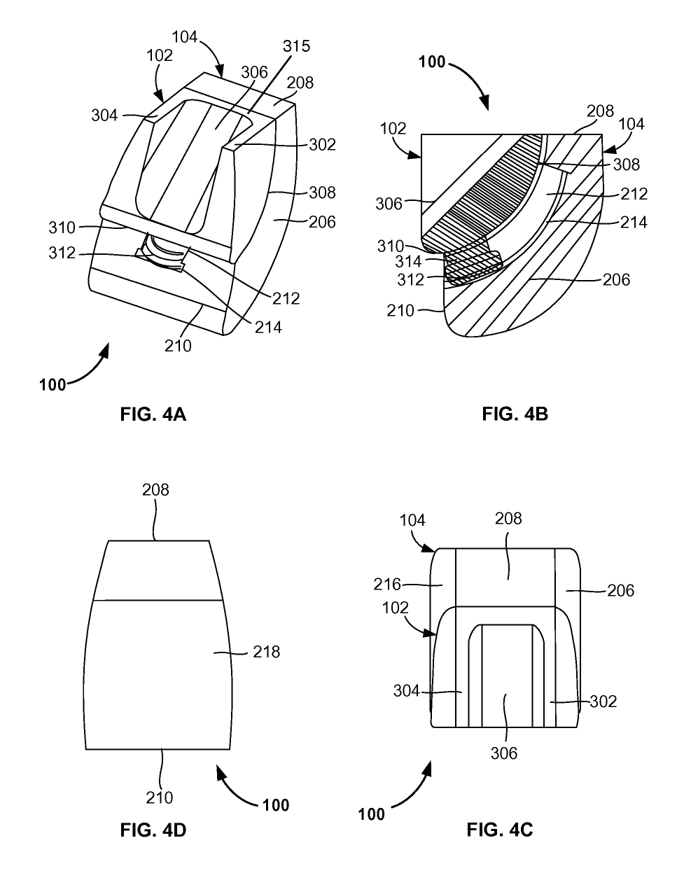

FIGS. 4A through 4D illustrate a perspective view, a sectional view, a top view, and a back view, respectively, of the sliding intervertebral implant 100 of FIG. 1 having the inner member 102 and the outer member 104 in a non-extended position according to embodiment herein. In FIGS. 4A through 4D, the top portion 208 of the first curved side 206 of the outer member 104 is in the same plane as that of a top 315 of the first arm 302 and a top 315 of the second arm 304 of the inner member 102 (i.e., planar top surfaces). FIG. 4A is the perspective view of the sliding intervertebral implant 100. As shown, the first arm 302 and the second arm 304 of the inner member 102 may be curved at their back side 308.

The curved back 308 matches with the curved shape of the first curved arm 202 and the second curved arm 204 of the outer member 104. FIG. 4B is the sectional view of the sliding intervertebral implant 100. The knob 312 with the neck 314 present at the inner bottom portion 310 of the inner member 102 may fit into the channel 212 of the outer member 104. The knob 312 is coupled into the extension 214 and the neck 314 fits into the channel 212. FIG. 4C is the top view which shows the first arm 302, the second arm 304, and the inclined plane 306 of the inner member 102. The outer member 104 includes the first curved side 206 having the top portion 208 and the second curved side 216. FIG. 4D is the back view which shows the top portion 208, the outer bottom portion 210, and the back side 218 of the outer member 104.

The fitting mechanism of the knob 312 in the extension 214 prevents decoupling of the inner member 102 from the outer member 104. The channel 212 accommodates the inner member 102 to slide with respect to the outer member 104 within a limitation of pre-set curvature. This curvature may follow a pattern of the outer shape of the intervertebral space. When the sliding intervertebral implant 100 is inserted into the intervertebral space, it is in the non-extended position in which the final surface contact of the implant 100 may not be changed; however the implant 100 may slide afterwards to sustain forces from the adjacent vertebrae.

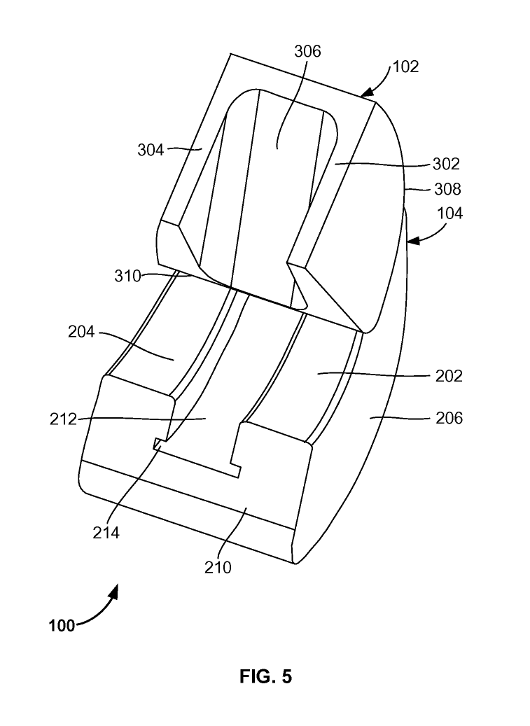

FIG. 5 illustrates a perspective view of the sliding intervertebral implant 100 of FIG. 1 having the inner member 102 and the outer member 104 in an extended position according to an embodiment herein. In this extended position the inner member 102 may be in its maximum sliding position. The first curved arm 202 and the second curved arm 204 support the curved back 308 of the inner member 102. The inner member 102 slides towards the top portion 208 (of FIGS. 2A through 2D) of the outer member 104. The knob 312 with the neck 314 (of FIGS. 3A, 3B, and 3D) of the inner member 102 is coupled to the extension 314 and the channel 212 of the outer member 104, respectively. The end of the channel 212 near the top portion 208 of the outer member 104 (as shown in FIGS. 2A, 2B, and 4B) act as a stop for the movement of the knob 212 and, correspondingly, the inner member 102. In other words, the inner member 102 does not slide past the second end 217 of the channel 212. After the inner member 102 and the outer member 104 are assembled, the open end 215 of the channel 212 at the outer member 104 may be pinned using pinning means (not shown) to ensure that the knob 312 is not disengaged from the outer member 104. The pinning means may include a clamp, a pin, a screw, or any other known mechanism suitable for retaining the inner member 102 to the outer member 104. Additionally, the pinning means may include a catch or other wall-like member that is used to block the open end 215 of the channel 212 after the inner member 102 and outer member 104 are assembled thereby preventing the inner member 102 from sliding out of the outer member 104.

The knob 312 acts as a fitting mechanism and helps the inner member 102 to slide over the outer member 104. The fitting mechanism of the sliding intervertebral implant 100 may accommodate the inner member 102 and the outer member 104 to slide with respect to each other within a limitation of the pre-set curvature. This curvature may follow a pattern of the outer shape of the intervertebral space (i.e., space between two adjacent vertebrae). When the sliding intervertebral implant 100 is inserted into the intervertebral space, the final surface contact of the implant 100 may not be changed (e.g., the non-extended position of the sliding intervertebral implant 100 of FIGS. 4A through 4D). However, at the final stage, the inner member 102 may fully slide and form the extended position (e.g., an arcuate structure).

This extended position of the implant 100 may increase a length of supporting surface between two adjacent vertebrae by increasing a length of the intervertebral implant 100. It may sustain forces from the adjacent vertebrae and increase stability of the vertebral column. The curvature of the sliding intervertebral implant 100 helps in increasing the length of supporting surface between the adjacent vertebrae since the changes in length affects the moment of inertia, the torsion value in lengthened surface is also affected greatly.

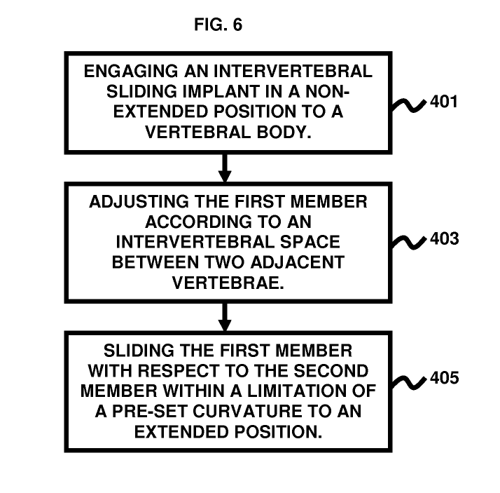

FIG. 6, with reference to FIGS. 1 through 5, is a process flow that illustrates a method of performing a surgical procedure according to an embodiment herein, wherein the method comprises engaging (401) an intervertebral sliding implant 100 in a non-extended position to a vertebral body (not shown), wherein the intervertebral sliding implant 100 comprises a first member 102 and a second member 104 slidably attached to one another; adjusting (403) the first member 102 according to an intervertebral space (not shown) between two adjacent vertebrae (not shown); and sliding (405) the first member 102 with respect to the second member 104 within a limitation of a pre-set curvature to an extended position, wherein the pre-set curvature is based on a pattern of a second shape of the intervertebral space.

Preferably, the first member 102 of the intervertebral sliding implant 100 comprises an inclined plane 306 adapted to fit a second shape of the intervertebral space; a curved back 308 coupled to the inclined plane 306; a knob 312 coupled to the curved back 308; and a neck 314 coupled to the knob 312 and the curved back 308 of the first member 102. Furthermore, the second member 104 of the intervertebral sliding implant 100 preferably comprises a first curved arm 202; a second curved arm 204, wherein the first curved arm 202 and the second curved arm 204 are configured to accommodate the curved back 308 of the first member 102; and a channel 212 separating the first curved arm 202 and the second curved arm 204, wherein the knob 312 and the neck 314 slide in the channel 212.

Additionally, the first member 102 and the second member 104 may be adapted to slide in an arcuate path with respect to one another from a non-extended position to an extended position. Moreover, the channel 212 of the second member 104 may comprise an extension 214 adapted to accommodate the knob 312 of the first member 104. Also, the channel 212 may comprise an open end 215 and a closed end 217, wherein the closed end 217 fixes an end point of the first member 102 to slide with respect to the second member 104.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A sliding intervertebral implant comprising:
an inner member adapted to connect to an intervertebral space between two adjacent vertebrae, wherein said inner member comprises:
a pair of arms spaced apart by a gap;
an inclined plane configured in said gap;
a back portion comprising an arcuate shape; and
a fitting mechanism coupled below said pair of arms and said arcuate back portion, wherein said fitting mechanism comprises a knob and a neck;
an outer member slidably attached to said inner member, wherein said outer member comprises a pair of curved arms spaced apart by a channel, wherein said pair of curved arms comprise an arcuate shape to match said arcuate shape of said back portion of said inner member,
wherein said knob is configured to slide in said channel within a limitation of a pre-set curvature, said pre-set curvature based on a pattern of a shape of said intervertebral space.

2. The sliding intervertebral implant of claim 1, all the limitations of which are incorporated herein by reference, wherein said channel comprises an open end and a closed end, wherein said closed end fixes an end point of said inner member to slide with respect to said outer member.

3. The sliding intervertebral implant of claim 1, all the limitations of which are incorporated herein by reference, wherein said knob comprises a width greater than a width of said neck.

4. The sliding intervertebral implant of claim 1, all the limitations of which are incorporated herein by reference, wherein said channel comprises areas of multiple widths, wherein said multiple widths comprise a first width and a second width, and wherein said second width is greater than said first width.

5. The sliding intervertebral implant of claim 4, all the limitations of which are incorporated herein by reference, wherein said knob is adapted to slide with respect to the second width area of said channel.

6. The sliding intervertebral implant of claim 1, all the limitations of which are incorporated herein by reference, wherein said inner member and said outer member are adapted to slide with respect to one another from a non-extended position to an extended position.

7. The sliding intervertebral implant of claim 6, all the limitations of which are incorporated herein by reference, wherein a top surface of said pair of arms of said inner member and a top surface of said outer member are planar with respect to one another in said non-extended position.

8. A sliding interbody device comprising:
a wedge-shaped first member adapted to connect to an intervertebral space between two adjacent vertebrae in a human body, wherein said first member comprises a pair of opposed arms and a knob, and
an arcuate shaped second member configured to slidably attach to said first member, wherein said second member comprises a pair of opposed curved arms having a channel disposed therebetween,
wherein each opposed curved arm comprises an arcuate top surface configured to match a contour of said first member,
wherein said knob is configured to slide in said channel within a limitation of a pre-set curvature, said pre-set curvature based on a pattern of a shape of said intervertebral space, and
wherein said channel comprises an open end and a closed end, wherein said closed end fixes an end point of said first member to slide with respect to said second member.

9. The sliding interbody device of claim 8, all the limitations of which are incorporated herein by reference, wherein said pair of opposed arms of said first member comprises a first arm perpendicular to a second arm, and wherein said first member further comprises:
a gap separating said first arm from said second arm;
an inclined plane configured in said gap and extending the length of said gap;
a back portion comprising an arcuate shape;
a bottom portion comprising an arcuate shape; and
a neck outwardly extending from said bottom portion,
wherein said knob outwardly extends from said neck, and
wherein said knob is diametrically larger than said neck.

10. The sliding interbody device of claim 8, all the limitations of which are incorporated herein by reference, wherein said channel comprises areas of multiple widths, wherein said multiple widths comprise a first width and a second width, and wherein said second width is greater than said first width.

11. The sliding interbody device of claim 8, all the limitations of which are incorporated herein by reference, wherein said pair of opposed curved arms of said second member comprises a first curved arm perpendicular to a second curved arm, wherein said channel comprises an arcuate shape and separates said first curved arm from said second curved arm, and wherein said channel comprises a pair of diametrically opposed grooves extending the length of said channel and located at a bottom portion of said channel.

12. The sliding interbody device of claim 11, all the limitations of which are incorporated herein by reference, wherein said knob is adapted to align with said grooves of said channel.

13. The sliding interbody device of claim 8, all the limitations of which are incorporated herein by reference, wherein said first member and said second member are adapted to slide in an arcuate path with respect to one another from a non-extended position to an extended position.

14. The sliding interbody device of claim 13, all the limitations of which are incorporated herein by reference, wherein a top surface of said pair of opposed arms of said first member and a top surface of said second member are planar with respect to one another in said non-extended position.

15. A method of performing a surgical procedure, said method comprising:
engaging an intervertebral sliding implant in a non-extended position to a vertebral body, wherein said intervertebral sliding implant comprises a first member and a second member slidably attached to one another;
adjusting said first member according to an intervertebral space between two adjacent vertebrae; and
sliding said first member with respect to said second member within a limitation of a pre-set curvature to an extended position,
wherein said pre-set curvature is based on a pattern of a second shape of said intervertebral space.

16. The method of claim 15, all the limitations of which are incorporated herein by reference, wherein said first member of said intervertebral sliding implant comprises:
an inclined plane adapted to fit a second shape of said intervertebral space;
a curved back coupled to said inclined plane;
a knob coupled to said curved back; and
a neck coupled to said knob and said curved back of said first member.

17. The method of claim 16, all the limitations of which are incorporated herein by reference, wherein said second member of said intervertebral sliding implant comprises:
a first curved arm;
a second curved arm, wherein said first curved arm and said second curved arm are configured to accommodate said curved back of said first member; and
a channel separating said first curved arm and said second curved arm,
wherein said knob and said neck slide in said channel.

18. The method of claim 16, all the limitations of which are incorporated herein by reference, wherein said first member and said second member are adapted to slide in an arcuate path with respect to one another from a non-extended position to an extended position.

19. The method of claim 17, all the limitations of which are incorporated herein by reference, wherein said channel of said second member comprises an extension adapted to accommodate said knob of said first member.

20. The method of claim 17, all the limitations of which are incorporated herein by reference, wherein said channel comprises an open end and a closed end, wherein said closed end fixes an end point of said first member to slide with respect to said second member.
